(19) United States
(12) Patent Application Publication
(10) Pub. No.: US 2010/0065070 A1
(43) Pub. Date: Mar. 18, 2010

(54) SAFETY BANDAGE WITH THIGH CUFFS

(76) Inventors: Alexander Sanchez, Jeseburg (DE); Roman Wysozki, Hamburg (DE)

Correspondence Address: KELLY LOWRY & KELLEY, LLP, 6320 CANOGA AVENUE, SUITE 1650, WOODLAND HILLS, CA, 91367, US

(21) Appl. No.: 12/304,358

(22) PCT Filed: Jun. 13, 2007
(86) PCT No.: PCT/EP07/05199

§ 371 Date: Aug. 18, 2009

(30) Foreign Application Priority Data
Jun. 14, 2006 (DE) 20 2006 009 397.4

(51) Int. Cl. A61F 5/37

(52) U.S. Cl. 128/876

(57) ABSTRACT

A safety bandage is provided for limiting the mobility of a patient lying on a support, or for positioning a patient on a support, with a retention strap to which a body strap enclosing the patient's body in the abdominal area is movably connected, and with thigh cuffs which enclose the patient's thighs. To improve a safety bandage of this kind in such a way that a patient cannot independently pull off the thigh cuffs, but without constricting the patient too much, the safety bandage has two leg fixture straps which are arranged transverse to the body strap and which connect the body strap and the thigh cuffs, wherein a material surface is formed between the leg fixture straps or material crosspieces are provided.

### TECHNICAL FIELD

The invention relates to a safety restraint for limiting the mobility of a patient lying on a support, or for positioning a patient on a support, comprising a retention strap to which a body strap enclosing the patient's body in the abdominal area is movably connected, and comprising thigh cuffs which enclose the patient's thighs.

### PRIOR ART

Such safety restraints are known and familiar to the person skilled in the art in very diverse embodiments and are described, for example, in DE 296 01 701 U1.

With the body strap slung around the abdominal region of the patient, the mobility of the patient is restricted insofar as he cannot leave the couch to which the body strap is fastened by means of leg fixture straps against the wishes of the supervisory person. In order to fix the body strap relative to the body of the patient thereon, without needing to tighten the body strap too tightly, which could result in impairment of the respiratory breathing of the patient, it is known from DE 203 17 701 U1 to provide additional thigh cuffs on the body strap or the safety restraint by means of retention straps aligned transversely thereto. In this case, each of the two thighs of the patients are encompassed by one of the thigh cuffs assigned thereto, which are provided with suitable closure means for adjusting the thigh circumference of the patient. These thigh cuffs ensure that the patient can neither intentionally or unintentionally wriggle downwards out of the safety restraint or its body strap and leave the support, for example, a bed or a specially provided patient trolley. In addition, a strap can be provided which runs transversely to the body strap through the crotch of the patient. However, this can lead to crushing of the patient's genitals if an attempt is made to wriggle out of the strap. Likewise, self-strangulation of the patient with this strap is not excluded.

However, in the case of such safety restraints with leg fixture and thigh cuffs, it is possible than an agile patient can draw his knee towards the upper body so that the relative distance between the body strap and the thigh cuffs is reduced. With the knee's drawn up, the patient can therefore slip the thigh cuffs over his knee since the leg fixture straps are loose.

### DESCRIPTION OF THE INVENTION, OBJECT, SOLUTION, ADVANTAGES

It is therefore the object of the invention to improve a generic safety restraint in such a manner that a patient cannot independently slip off the thigh cuffs without excessively constricting him, in particular, any lateral pulling or turning of the thigh cuffs so as to be able to slip them off should be prevented.

This object is achieved by the features of claim 1.

The basic idea of the invention is that a material surface is formed between the leg fixture straps. The function of a material surface holding the bottom can also be achieved with several material crosspieces but it should be noted that one material crosspiece is not sufficient. In this way it is prevented that the leg fixture straps can be pulled laterally outwards and thus the patient could slip the thigh cuffs allocated to the leg fixture straps over his knee. For example, if the patient only tucks up one leg to slip off that leg's thigh cuff, the material crosspieces or the material surface have the effect that this tucking up or bending is only possible to a certain degree since by means of the material crosspieces or the material surface, the other leg is also pulled towards the body by means of the leg fixture strap and the thigh cuff. However, if both legs are tucked up at the same time, so that the material crosspieces or the material surface are not set under tensile stress, it is not possible to slip off the thigh cuff since, due to the leg fixture straps, the maximum possible distance of the thigh cuff from the body strap is predetermined and slipping off is prevented. When the legs are tucked up, the material crosspieces or the material surface exert a gentle pressure on the patient's thighs or bottom so that the patient makes no further attempts to slip off the thigh cuff. The additional material crosspieces or the material surface ensure that the distance between the two leg fixture straps cannot be enlarged beyond the length predetermined by the material crosspieces or the material surface so that the patient, for example, cannot slide his bottom between the leg fixture straps to slip off the thigh cuff.

The exact position and width of the material crosspieces and the material surface or their distance from the thigh cuffs and from the body strap can be selected by the person skilled in the art to effectively prevent any slipping-out.

The material of the crosspieces and the surface corresponds to the known materials of such safety restraints and is preferably the same as the material of the body strap, the leg fixture straps or the thigh cuffs.

The advantage of the invention is that with the thigh cuffs, it is not possible for the patient to independently free himself from the safety restraint, wherein the thigh cuffs in particular exert no pressure on the patient's genitals and crushing of the genitals is avoided since no additional strap is required through the crotch. The additional material crosspieces or the material surface additionally prevent the patient from being able to slip off the thigh cuffs either successively or simultaneously. The thigh cuffs also prevent the patient from sliding down in the body strap and in such a manner that the body strap exerts an excessively large pressure on the patient's thoracic cage or there is a risk of self-strangulation for the patient. In addition, the patient retains a relatively large measure of freedom of movement since a diagonal fixing otherwise necessary to avoid self-strangulation is not provided.

Another major advantage of this safety restraint according to the invention in contrast to trousers, jacket-like and sleeping-bag-like constructions is that no unnecessary warming or overheating of the patient occurs since only straps are in fact attached, which allow sufficient ventilation area.

Advantageous embodiments of the invention are characterised in the dependent claims.

In a preferred embodiment, two additional longitudinal straps are disposed on an upper side of the safety restraint or on its body strap, each of said straps being connected to the thigh cuffs. These additional longitudinal straps are likewise aligned substantially perpendicularly to the body strap and connect these to the thigh cuffs in the applied state of the safety restraint, running along the upper side of the patient's thighs. These longitudinal straps prevent any enlargement of the distance between the thigh cuffs and the body strap on the upper side, in order to maintain their relative distance from one another.

According to a particularly advantageous embodiment, a second crosspiece is provided for the additional longitudinal straps, said crosspiece being fastened, for example, sewn onto the longitudinal straps and, in the applied state of the safety restraint, resting on the cuter sides of the patient's thighs and encompassing these completely circumferentially from behind. In addition, the second crosspiece is connected to the leg fixture straps. The second crosspiece is interrupted on the upper side between the longitudinal straps. It is thus avoided that the top-side longitudinal straps can be drawn or moved towards one another, towards the middle, i.e. towards the symmetry plane of the safety restraint to undesirably reduce the distance between the thigh cuffs and the body strap. This is avoided by the continuous crosspiece running on the back of the patient's thigh and thus the longitudinal straps cannot be pulled together. Lateral slippage of a longitudinal strap inwards is prevented by the tension transmitted by the second crosspiece to the ether longitudinal strap. In addition, the leg fixture straps are additionally fixed in their position with the second crosspiece. The continuously running second crosspiece also exerts a gentle pressure on the thighs when these are jointly tucked up so that the patient cannot slip off the thigh cuffs either singly or jointly.

In addition, shoulder straps known to the person skilled in the art can be provided on the safety restraint, which are likewise aligned substantially transversely to the body strap or are arranged so that they cross, and are guided over the patient's shoulders and fastened to the body strap. Jointly with such shoulder retainers, it is almost impossible for the patient to wriggle independently out of the safety restraint. Slipping off the thigh cuffs is effectively prevented by the shoulder straps attached fixedly or detachably to the body strap. It is not possible for the patient to pull the body strap and thus the thigh cuffs fastened thereon downwards and then slip it off. Any attempt to slip off the thigh cuffs by bending the legs and/or contorting the back is prevented by means of the tension coming from the shoulder straps via body strap and leg fixture straps. The safety restraint is therefore based on a mechanism in such a manner that when an attempt is made to reduce the distance between the thigh cuffs and the knee by bending the legs and/or contorting the back, the distance is in turn enlarged in a quasi-self-regulating manner by means of the tension introduced by the shoulder straps over the back and bottom, which effectively prevents the thigh cuffs from being slipped off. The longitudinal strap and the crosspieces or the material surface thereby prevent the evasion or bypassing of this mechanism so that the patient is impeded from slipping out downwards by means of the thus non-removable thigh cuffs without the risk of strangulation or crushing the genitals and is prevented from slipping out upwards by means of the shoulder straps and ultimately remains in the safety restraint. It is understood that the shoulder retainers can either be fastened detachably, e.g. with loops on the body strap or retention strap or they are disposed fixedly thereon, preferably sewn with this. Such shoulder retainers can also be configured as a retrofit kit for already provided body straps, which are additionally fastened to this to increase the safety.

In order to increase the wearing comfort of the safety restraint and avoid pressure points, the material surface and the material crosspieces are made of material or fabric. In the same way, a mesh of materials suitable for this purpose can take over the function of the crosspieces. It is also comprised within the scope of the invention that the first and the second crosspiece and optionally the thigh cuffs are configured in one piece with one another and are continuous and flat. In this case, when tucking up the thighs, such a flat design would exert a pressure on the bottom and the back of the patient's thighs so that any independent slipping off of the thigh cuffs is eliminated. In this case, the body strap, a shoulder retainer described hereinafter and the crosspiece or the mesh between the leg fixture straps form a unit. If the patient tucks up one or both knees, a tension is exerted on the thigh cuffs in the direction of the patient's upper body by means of the mesh in conjunction with the shoulder straps, so that the patient cannot slip this off over the knee.

In order to retrofit already provided safety restraints with thigh cuffs which are secured against slipping off, the leg fixture straps on the body strap or on the retention strap and optionally the longitudinal straps on the upper side of the safety restraint are detachably fastened on the body strap. The person skilled in the art can use for this purpose all the fastening means known in the prior art such as eyelets with magnetic buttons, strap locks, Velcro closures, tabs and loops or the like. It is thus possible to retrofit a safety restraint substantially consisting of a body strap, with the thigh cuffs, whereby undesirable slipping off is prevented by the additional crosspieces. Such a retrofit kit can be firmly sewn with an existing body strap.

It is advantageous if the leg fixture straps and the shoulder straps as well as the longitudinal straps are formed in one piece, wherein the straps formed in one piece can be passed through a pass-through pocket located on the body strap or the retention strap. In this way, the distance between body strap and thigh cuffs can be configured to be variable. This purpose is also served by the strap formed in one piece having eyelets and/or pockets and/or loops. The straps formed in one piece are then pulled through the pass-through pocket and fastened with locks at the ends of the body strap and the thigh cuffs. This can be achieved in such a manner that the straps formed in one piece are guided over the body strap parallel to the thigh cuffs and cross over above the body strap.

The ends on the thigh cuffs, which serve as bed retainers, can preferably be connected diagonally to the ends of the retention strap in a fixed or detachable manner. In this way, the patient is prevented from turning about the vertical axis.

The body strap is preferably fixedly connected to a shoulder retainer, the shoulder retainer being provided with two shoulder straps which run over the shoulders of the patient and are connected to the waist strap in the central region of the waist strap, wherein the waist strap can encompass the waist of the patient and both ends of the waist strap with respectively two shoulder straps, these in turn connecting to one another, are detachably connected to one another, and strap locks which are provided with a locking position to be opened with suitable closure means, and the ends of the waist strap on the shoulder straps are connected to one another in pairs. In this way it is achieved that a firmer fit of the shoulder retainer on the body strap or retention strap is achieved compared to a shoulder retainer detachably attached on the body strap or retention strap. A shoulder retainer according to the prior art can also be fixedly or detachably connected to the body or retention strap.

A further embodiment of the invention provides that at least one additional crosspiece is provided between the two shoulder straps disposed substantially transversely to the body strap, the crosspiece being aligned substantially parallel to the body strap. This additional crosspiece prevents the shoulder straps from being slipped out sideways and being slipped off over the shoulders by the patient since during a lateral displacement of one of the shoulder strap a tension is transmitted to the outer shoulder strap via the crosspiece and this is pulled towards the first shoulder strap so that this for example makes contact with the patient's neck and prevents any further lateral displacement of the first shoulder strap. Likewise, the crosspiece prevents both shoulder straps from being pulled apart from one another simultaneously to the side or outwards. The additional crosspiece ensures that the distance between the two shoulder straps cannot be enlarged beyond the length predetermined by the crosspiece so that the patient cannot, for example, wriggle his shoulder out of one of the straps to slip off the shoulder straps.

It is comprised within the scope of the invention that the two shoulder straps are aligned substantially perpendicularly to the body strap and parallel to one another as well as a crossover arrangement of the shoulder straps.

The precise position, that is the distance from the body strap and the width of the crosspiece, can be selected by the person skilled in the art to effectively prevent the shoulder straps slipping off.

It is understood that more than one crosspiece which are each aligned substantially transversely to the body strap can be provided between the shoulder straps arranged approximately parallel to one another. For example, a further crosspiece can be provided on the front side of the safety restraint approximately in the chest area of the patient, which crosspiece can advantageously be detachably fastened at least on one shoulder strap for easier application of the safety restraint. The material of the crosspiece corresponds to the known materials of such safety restraints and is preferably the same as the material of the body straps or the shoulder straps.

The advantage of this embodiment is that with the shoulder straps, the patient is effectively prevented from freeing himself from the safety restraint, although his freedom of movement is only insignificantly restricted by the additional shoulder straps which are connected amongst one another to a crosspiece.

In another embodiment, the shoulder straps are disposed on an additional waist strap which for its part is aligned substantially parallel to the body strap. The waist strap is provided for looping around the waist of the patient and can be adjusted to the waist size of the patient with suitable closure means. The shoulder straps are preferably fastened on the back or underside of the safety restraint on the waist strap and are guided from behind over the patient's shoulders and fastened in a suitable manner at the front either on the waist strap or on the body strap. In particular, it is proposed that loops are provided on the body strap through which the additional waist strap is passed so that the waist strap is movable relative to the body strap or can be displaced slightly laterally.

In one alternative embodiment, the shoulder straps which are aligned substantially parallel to one another are fastened directly on the body strap or the retention strap and are guided over the shoulders of the patient and fixed with suitable closure means on the front side of the body strap.

In this embodiment, a further breast strap can be provided on the shoulder straps which is fastened on the back of the patient's body on the shoulder straps and is aligned substantially transversely to the body strap. This breast strap encompasses the chest area of the patient and can also be fixed on the front of the patient on the shoulder straps guided over the shoulders, for example, on tabs or loops formed thereon.

The crosspiece or crosspieces between the shoulder straps are preferably designed to be flat to achieve a large contact area in this manner so that no pressure points are produced on the patient's skin due to the safety restraint. In this case, the crosspieces can be designed to be flat in the form of a material or fabric made of a material known for safety restraints or as a mesh made of a material suitable for this purpose.

In order to retrofit already provided safety restraints with shoulder straps, which are secured against slipping off, the shoulder straps are fastened detachably on the body strap or on the retention strap. The person skilled in the art can use for this purpose all fastening means known in the prior art, such as eyelets with magnetic buttons, strap locks, Velcro closures, tabs and loops or the like. It is thus possible to retrofit a safety restraint which substantially consists of a body strap, with the shoulder straps, wherein the additional crosspieces prevent undesirable slipping off. Such a retrofitting kit can also be firmly sewn to an existing body strap.

It is advantageous if the two retention straps can be detachably fastened to a foot end of the bed frame. It is also within the scope of the invention that at least one extension strap is fastened on the crosspiece and/or on the leg fixture straps and/or the retention strap and/or body strap and can be detachably fastened to a foot end of the bed frame. A practical variant of the invention additionally provides that extension straps which can be detachably fastened to the head end of the bed frame can be fastened on the shoulder straps and/or retention strap and/or body strap and/or crosspieces.

### BRIEF DESCRIPTION OF THE DRAWINGS

Exemplary embodiments of the invention are explained in detail hereinafter with reference to the drawings. In the figures in perspective view:

FIG. 1: shows a safety restraint;

FIG. 2: shows one embodiment of the safety restraint according to the invention in which a shoulder retainer is detachably fastened;

FIG. 3: shows another embodiment with integrated shoulder retainer; and

FIG. 4: shows another embodiment of the safety restraint according to the invention.

### BEST MODE FOR CARRYING OUT THE INVENTION

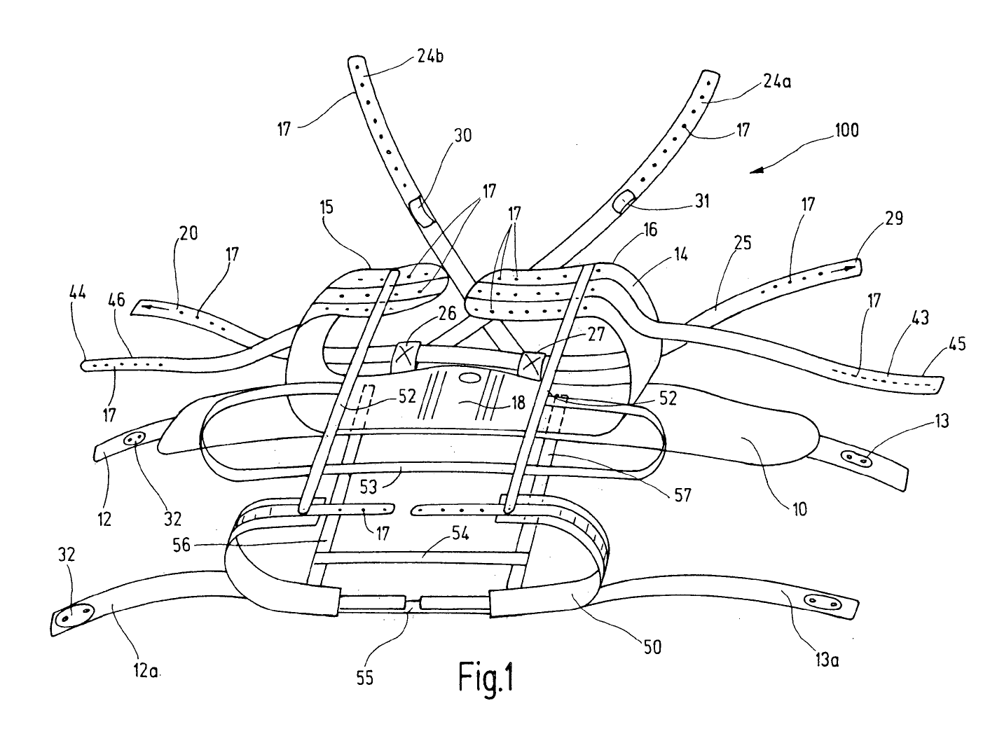

The safety restraint 100 shown in FIG. 1 substantially consists of a retention strap 10 which is placed directly on a support, not shown here, so that it crosses thereover, and which is fastened to the support by means of fastening means provided at the ends 12, 13, for example, eyes 32. Furthermore, the safety restraint 100 has a body strap 14 which is movably connected to the retention strap 10 in a manner likewise not shown. The ends 15, 16 of the body strap 14 each have three rows of eyelets 17 which are aligned parallel in the longitudinal direction of the ends 15, 16. In principle, only one or two rows of eyelets 17 can also be provided. At the centre, the body strap 14 has a pass-through pocket 18 through which a multimembered strap in accordance with DE 203 17 701 U1 can be passed transversely to the body strap 14 but this is no longer necessary in the configuration of the safety restraint 100 according to the invention.

In addition, further fastening straps 43, 44 can be provided on the body strap 14, whose free ends 45, 46 are each provided with eyelets 17 for fastening, for example, on the support or on a bed frame. The fastening straps 43, 44 can also be fastened detachably on the body strap 14.

For encompassing the shoulders of a patient, crossing-over shoulder straps 24a, 24b with eyelets at their free ends are provided on an additional strap 25. The strap 25, which runs substantially parallel to the body strap 14 is passed through loops 26, 27 on the body strap 14 and at its free ends 28, 29 also has eyelets 17 in order to loop and fix the strap 25 around the patient's waist. At the same time, the strap 25 can be guided through a loop 30 which is disposed on the section 24b. A pocket 31 is disposed on the section 24a for handling, for example, magnetic closures on the strap 25.

In addition, the safety restraint 100 is provided with two thigh cuffs 50 having broad cuff-like sections, likewise having eyelets 17 disposed on their one free and narrower end so that this section of the thigh cuff 50 can be looped through a metal ring not shown at the other free end to completely encompass a patient's thigh. The eyelets 17 are fixed for example, with magnetic buttons or with other known connecting means. The thigh cuffs 50 are each disposed on a leg fixture straps 56; 57 which are aligned substantially perpendicularly to the body strap 14 and are fastened thereon. A crosspiece 54 is disposed between the leg fixture straps 56, 57 to prevent the patient from independently tucking up or bending only one leg or thigh. It is thus prevented that when a knee is tucked up to the patient's chest, the patient cannot slip off the thigh cuff 50 over the knee and the lower leg independently of the thigh. Further ends 12a, 13a with eyes 32 corresponding to the retention strap 10, can be provided on the thigh straps 50 for fastening on the support.

In addition, further longitudinal straps 52 are provided on the upper side of the safety restraint 100 which run substantially from the body strap 14 in each case to a thigh cuff 50 and are also aligned transversely to the body strap 14. The longitudinal straps 52 are connected continuously to a second crosspiece 53 running around the back of the safety restraint 100. It is thus avoided that the longitudinal straps 52 which are likewise aligned substantially perpendicularly to the body strap 14, can be pulled together e.g. towards the centre of the safety restraint 100 and a thigh cuff 50 can thus undesirably be slipped off. In addition, a third crosspiece 55 is provided in the area of the thigh cuff 50.

The thigh cuffs 50 and the crosspieces 53, 54, 55 are dimensioned in such a manner that they form no pressure points on the patient's skin. For connection of the different straps and crosspieces to one another, these are each sewn and/or bonded at the contact points or connected to one another with other connecting means known for safety restraints 100.

In order to retrofit a safety restraint 100 with thigh cuffs 50, the longitudinal straps 52 and the leg fixture straps 56, 57 are detachably fastened on the body strap 14 or the retention strap 10, for example, at the eyelets 17 of the body strap 14 and can also be provided as a retrofit kit for an already existing safety restraint 100.

The crosspieces 53, 54, 55 can also be configured flat or mesh-shaped or they are dimensioned and arranged in such a manner that they jointly fulfil the functionality of a flat form in order to thereby exert a gentle pressure on the bottom and the back of the patient's thighs when the legs or the thighs are tucked up so that the patient cannot tuck up his legs since at the same time the crossing-over sections 24a, 24b exert a tensile force in the opposite direction on the body strap 14 or the thigh cuffs 50. The formation of pressure points is therefore effectively avoided. The thigh cuffs 50 can, for example, be sewn onto the crosspiece 55.

The function of the second crosspiece 53 is primarily to connect the leg fixture straps 56, 57 or the material surface located therebetween to the longitudinal straps 52 so as to prevent any lateral pulling or twisting of the thigh cuffs 50 to slip these off. If the function of the material surface holding the bottom is achieved with crosspieces 54, 55, the connecting straps between the leg fixture straps 56, 57 and longitudinal straps 52 can be achieved with strap 53 which at the same time serves as a cross-strap.

The slipping off of the thigh cuffs 50 can be effectively prevented by means of shoulder straps 24a, 24b attached fixedly or detachably to the body strap 14 in that the patient cannot pull the body strap 14 and thus the thigh cuffs 50 fastened thereon downwards and then slip these off. Thus, any attempt to slip off the thigh cuffs by bending the legs end/or contorting the back is prevented by the tension originating from the shoulder straps 24a, 24b via body strap and leg fixture straps. A contributory factor is that the longitudinal strap 52 and the second crosspiece 53 and the first crosspiece and the further crosspiece 55 or the material surface prevent evasion of the mechanism described so that the patient is prevented from wriggling out downwards by means of the non-removable thigh cuffs without the risk of strangulation or crushing the genitals and is prevented from wriggling out upwards by means of the shoulder straps 24a, 24b and ultimately remains in the safety restraint. The operating mode of the mechanism therefore consists in that, when an attempt is made to reduce the distance from the thigh cuff 50 to the knee by bending the legs and/or contorting the back, the distance is enlarged in a quasi-self-regulating manner by means of the tension introduced by the shoulder straps 24a, 24b via back and bottom, with the consequence that slipping off of the thigh cuffs is effectively prevented.

Furthermore, the distance between body strap 14 and thigh cuffs 50 can be configured to be variable by attaching a pass-through pocket 18 on the body strap 18 through which the leg fixture straps 56, 57 are drawn. However, the latter are lengthened by the lengths of the body strap 14, the shoulder straps 24a, 24b and the longitudinal straps 52 and accordingly fitted with eyes and loops so that they take over the function of these straps. These straps now configured in one piece are therefore drawn through the pass-through pocket 18 and fastened with locks at the ends 15, 16 of the body strap 14 and the thigh cuffs 50. This is effected in such a manner that the straps configured in one piece are guided over the body strap 14 parallel to the thigh cuffs 50 and cross over the body strap 14. So that the straps cannot be slipped off over the shoulders, they are sewn together according to the shoulder restrains at the height of the shoulder blades and are provided with loops at different heights below the axilla so that the waist strap 28, 29 can be pushed through and connected to the straps configured in one piece with locks on the front. The straps configured in one piece can be firmly sewn to the body strap 14.

The ends 12a, 13a (not shown) i.e. the bed retainers on the thigh cuffs, can also be connected diagonally to the ends 12, 13. In this way, twisting of the patient about the vertical axis is prevented. Furthermore, the distance between body strap 14 and thigh cuff 50 designed to be so short that the strap 53 is not necessarily needed (not shown). The ends 12a, 13a can also be designed as a bed connecting strap for fastening to the support (not shown).

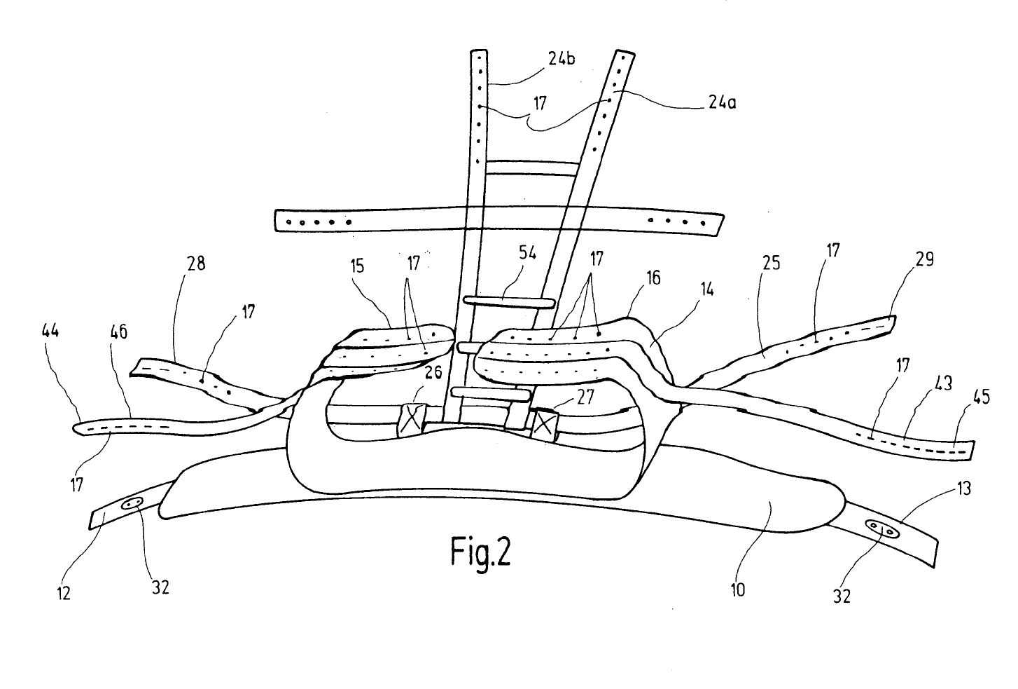

The embodiment of the safety restraint 100 without leg fixture straps 56, 57 shown in FIG. 2 substantially consists of a retention strap 10 which is placed directly on a support not shown here, crossing over this, and is fastened to the support by means of fastening means such as, for example, eyes 32 provided at the ends 12, 13. The safety restraint 100 also has a body strap 14 which is movably connected to the retention strap 10 in a manner likewise not shown. The ends 15, 16 of the body strap 14 each have three rows of eyelets 17 which are aligned parallel in the longitudinal direction of the ends 15, 16. In principle, only one or two rows of eyelets 17 can also be provided. The body strap 14 is adjusted and fixed to the abdominal girth of the patient by means of eyelets 17.

In addition, further fastening straps 43, 44 can be provided on the body strap 14 as side fastenings, whose free ends 45, 46 are each provided with eyelets 17 for fastening, for example, on the support or on a bed frame. The fastening straps 43, 44 can also be fastened detachably on the body strap 14.

For encompassing the waist of a patient, a waist strap 25 is provided on the safety restraint 100 which runs substantially parallel to the body strap 14. The waist strap 25 is passed through loops 26, 27 on the body strap 14 and at its free ends 28, 29 also has eyelets 17 in order to loop the waist strap 25 around the patient's waist and fix it, for example, with magnetic buttons.

Two shoulder straps 24a, 24b are fastened to the waist strap 25, for example, sewn and/or adhesively bonded, which, depending on the embodiment, are aligned substantially parallel or cross over one another and are guided over the shoulders of a patient. The shoulder straps 24a, 24b are also provided with eyelets 17 at their free ends to fix the shoulder straps 24a, 24b either on the waist strap 25 or on the body strap 14 at further eyelets 17 by means of suitable connecting means.

In order to prevent the patient from undesirably slipping off the shoulder straps 24a, 24b over his shoulders, one or more crosspieces 54 are provided between the shoulder straps 24a, 24b which are aligned substantially parallel to the body strap 14. The crosspiece or crosspieces 54 prevent the shoulder straps 24a, 24b from being pulled apart by the patients to slip these over the shoulders.

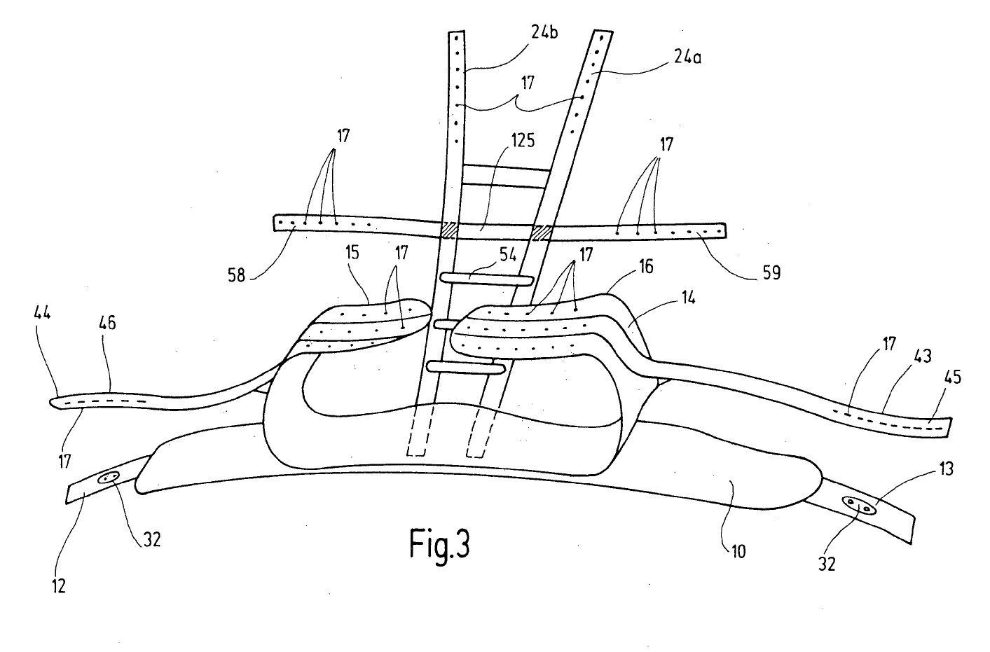

In the alternative embodiment without leg fixture straps 56, 57 shown in FIG. 3, the shoulder straps 24a, 24b are disposed directly on the body strap 14 or on the retention strap 10 and are in particular detachably fastened. The shoulder straps 24a, 24b are likewise connected to one another with crosspieces 54 to prevent any lateral pulling apart of the shoulder straps 24a, 24b. In addition, another breast strap is provided on the shoulder straps 24a, 24b to loop around the chest area of a patient. This breast strap 60 also acts as another crosspiece 54. At the free ends 126, 129 of the further breast strap 125, eyelets 17 are also provided for fixing the breast strap 60. The breast strap 60 can also be guided through loops not shown here on the shoulder straps 24a, 24b in front of the patient's chest.

The shoulder straps 24a, 24b, the breast straps 60 and the waist straps 25 as well as the crosspieces 54 are dimensioned so that no pressure points form on the patient's skin. For example, the crosspiece 54 can be configured to be flat or mesh-like. For connecting the different straps and crosspieces to one another, these are sewn and/or adhesively bonded or connected to one another by other connecting means known for safety restraints 100 in each case at their contact points.

In order to retrofit a safety restraint 100 with shoulder straps 24a, 24b, the shoulder straps 24a, 24b are detachably fastened to the body strap 14 or the retention strap 10, for example, at further eyelets 17 of the body strap 14 and can be supplied as a retrofit kit for an already existing safety restraint 100. The crosspieces 54 can also be designed to be flat or mesh-shaped sc that only a slight pressure is exerted on the patient's back without excessively adversely affecting him.

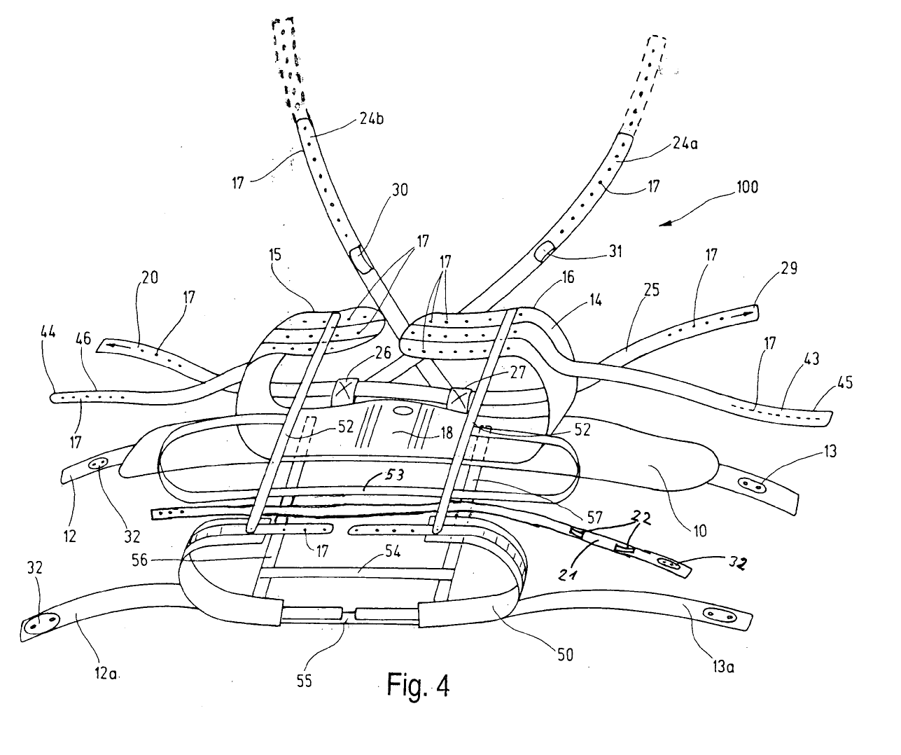

FIG. 4 shows another embodiment of the safety restraint according to the invention. In this embodiment the shoulder straps 24a, 24b are lengthened. A shoulder strap fixing 21 is disposed between body strap 14 and crosspiece 55 which can be guided over the patient's thigh and locked, in particular with a strap lock, and has loops 22 through which the shoulder straps 24a, 24b can be inserted. The loops 22 are in this case configured as pockets for strap locks in such a manner that the shoulder straps 24a, 24b can be fastened thereto.

### REFERENCE LIST

100 Safety restraint
10 Retention strap
12, 13 End
12a, 13a End
14 Body strap
15, 16 End
17 Eyelets
18 Pass-through pocket
21 Shoulder strap fixing
22 Loops
24a, 24b Shoulder straps
25 Strap
26, 27 Loop
28, 29 End
30 Loop
31 Pocket
32 Eye
43, 44 Retention straps
45, 46 End
50 Thigh cuff
52 Longitudinal strap
53 Second crosspiece
54 First crosspiece
55 Further crosspiece
56, 57 Leg fixture strap
58 Waist strap
59 Waist strap

1. A safety restraint for limiting the mobility of a patient lying on a support, or for positioning a patient on a support, comprising:
a retention strap to which a body strap enclosing the patient's body in the abdominal area is movably connected; and
thigh cuffs which enclose the patient's thighs;
characterised in that the safety restraint has two leg fixture straps which are arranged transversely to the body strap and which connect the body strap and the thigh cuffs, wherein a material surface is formed between the leg fixture straps or material crosspieces are provided.

2. The safety restraint according to claim 1, characterised in that two longitudinal straps are disposed on an upper side of the safety restraint, each of said straps being connected to a thigh cuff.

3. The safety restraint according to claim 2, characterised in that a second crosspiece (53) is provided for the longitudinal straps, said crosspiece likewise being connected to the leg fixture cuffs.

4. The safety restraint according to claim 1, characterised in that shoulder straps are provided on the safety restraint, said straps being fixed fixedly or detachably on the body strap.

5. The safety restraint according to claim 1, characterised in that the material surface and the material crosspieces are made of material or fabric and/or are formed as mesh.

6. The safety restraint according to claim 4, characterised in that the leg fixture straps and the longitudinal straps are detachably fastened on the body strap.

7. The safety restraint according to claim 6, characterised in that the leg fixture straps and the shoulder straps as well as the longitudinal straps are formed in one piece, wherein the straps formed in one piece can be passed through a pass-through pocket located on the body strap.

8. The safety restraint according to claim 7, characterised in that the strap formed in one piece has eyelets and/or pockets and/or loops.

9. The safety restraint according to claim 7, characterised in that the safety restraint has a waist strap which runs substantially parallel to the body strap, wherein the waist strap can be pushed through loops disposed on the shoulder straps.

10. The safety restraint according to claim 7, characterised in that the straps formed in one piece can be fastened with locks on the body strap and on the thigh cuffs.

11. The safety restraint according to claim 7, characterised in that the strap formed in one piece is sewn with the body strap.

12. The safety restraint according to claim 1, characterised in that the ends on the thigh cuffs which serve as a bed retainer can be connected diagonally to the ends of the retention strap.

13. The safety restraint according to claim 1, characterised in that the distance between body strap and thigh cuffs is shortened.

14. The safety restraint according to claim 3, characterised in that in the upper thigh region the second crosspiece is closable and encompasses the thighs of the patient in the upper area.

15. The safety restraint according to claim 14, characterised in that the crosspiece is closable by means of a connecting strap, wherein closure means are attached to the open ends, to which the connecting strap can be detachably fastened.

16. The safety restraint according to claim 4, characterised in that the body strap is fixedly or detachably connected to a shoulder retainer.

17. The safety restraint according to claim 16, characterised in that the shoulder retainer is provided with two shoulder straps which run over the shoulders of the patient and are connected to the waist strap in the central region of a waist strap, wherein the waist strap encompasses the waist of the patient and both ends of the waist strap with respectively two shoulder straps are detachably connected to one another, these shoulder straps in turn connected to one another, and strap locks which are provided with a locking position to be opened with suitable closure means, the ends of the waist belt and the shoulder straps are each connected to one another in pairs.

18. The safety restraint according to claim 17, characterised in that the shoulder straps are fastened on the waist strap by means of loops on the body strap.

19. The safety restraint according to claim 18, characterised in that a further breast strap is disposed on the shoulder straps.

20. The safety restraint according to claim 19, characterised in that the shoulder straps are detachably fastened on the safety restraint.

21. The safety restraint according to claim 1, characterised in that the at least one crosspiece is designed to be flat.

22. The safety restraint according to claim 4, characterised in that the leg fixture straps have spar retaining straps which encompass the thighs of the patient.

23. The safety restraint according to claim 22, characterised in that the spar retaining straps have channels through which the shoulder straps of a shoulder retainer can be inserted.

24. The safety restraint according to claim 16, characterised in that the shoulder retainer is designed as lengthened and can be connected to the thigh cuffs.

25. The safety restraint according to claim 1, characterised in that the leg fixture straps can be detachably fastened at a foot end of a bed frame.

26. The safety restraint according to claim 1, characterised in that at least one extension strap is or can be fastened on the crosspiece and/or on the leg fixture straps and/or retention strap and/or body strap and is configured in such a manner that it can be detachably fastened at a foot end of a bed frame.

27. The safety restraint according to claim 4, characterised in that extension straps are or can be fastened on the shoulder straps and/or retention strap and/or body strap and/or crosspieces, which can be detachably fastened at the head end of the bed frame.

28. The safety restraint according to claim 4, characterised in that the leg fixture straps and the shoulder straps as well as the longitudinal straps are formed in one piece, wherein the straps formed in one piece can be inserted into a pass-through tab located on the retention strap.

29. The safety restraint according to claim 7, characterised in that the strap formed in one piece is firmly sewn with the retention strap.

30. The safety restraint according to claim 11, characterised in that the ends on the thigh cuffs which serve as a bed retainer, can be fixedly or detachably connected fixedly or detachably to the ends of the retention strap.

31. The safety restraint according to claim 4, characterised in that the shoulder straps are lengthened.

32. The safety restraint according to claim 31, characterised in that a shoulder strap fixing is disposed between body strap and crosspiece, which can be guided around and encompass the thighs of the patient, in particular with a belt lock, and which has loops through which shoulder straps can be inserted.

33. The safety restraint according to claim 32, characterised in that the loops are formed as pockets for belt locks at which the shoulder straps can be fastened.

34. A retrofit kit for a safety restraint for limiting the mobility of a patient lying on a support, or for positioning a patient on a support, comprising a retention strap which can be placed fixedly on the support, extending over said support, to which a body strap enclosing the patient's body in the abdominal area is movably connected, wherein closure means are provided for the retention strap and for the body strap, comprising two leg fixture straps disposed transversely to the body strap, each having a thigh cuff, wherein a material surface or crosspieces are provided between the leg fixture straps and the leg fixture straps can be fastened detachably or non-detachably to the safety restraint.

35. The retrofit kit according to claim 34, characterised in that it is configured according to claim 1.
